(19) United States
(12) Patent Application Publication
(10) Pub. No.: US 2004/0191279 A1
(43) Pub. Date: Sep. 30, 2004

(54) Tacky skin care compositions and articles having tacky skin care compositions disposed thereon

(76) Inventor: Thomas James Klofta, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/402,820

(22) Filed: Mar. 28, 2003

(51) Int. Cl. A61K 7/00

(52) U.S. Cl. 424/401

(57) ABSTRACT

Skin care compositions which provide enhanced transfer of the composition to the skin and increased adhesion to the skin are disclosed. The skin care composition comprises:

a) from about 5% to about 95% by weight of an emollient,

b) from about 10% to about 95% by weight of a tackifying agent, and

c) optionally, from about 1% to about 95% by weight of an immobilizing agent.

Absorbent articles and other application devices comprising the skin care compositions are also disclosed.

### FIELD OF THE INVENTION

This invention is directed to a tacky skin care composition having improved adhesion to skin. Additionally disclosed are articles comprising this tacky skin care composition.

### BACKGROUND OF THE INVENTION

Skin is made up of several layers of cells, which coat and protect the keratin and collagen fibrous proteins that form the skeleton of its structure. The outermost of these layers, referred to as the stratum corneum, is known to be composed of 25 nm protein bundles surrounded by 8 nm thick layers. It is now recognized that maintaining the proper water gradient across the stratum corneum is important to its functionality. Most of this water comes from inside the body. If the humidity in the air surrounding the skin is too low, insufficient water remains in the outer layers of the stratum comeum to properly plasticize the skin. On the other hand, too much water on the outside of the skin causes the stratum corneum to ultimately sorb three to five times its own weight of bound water. This swells and puckers the skin and results in a two to three fold increase in the permeability of the skin to water and other polar molecules, which may become skin irritants.

Thus, a need exists for skin care compositions which will assist the stratum corneum in maintaining its barrier and water-retention functions in spite of external conditions. For maximum benefit, it is crucial that such skin care compositions be deposited onto the skin and then remain on the skin rather than being inadvertently removed. This enhanced skin transfer and retention efficiency is especially important when expensive skin care ingredients are included in the composition.

Wearable absorbent articles such as diapers are well known in the art. Absorbent articles typically have an absorbent core, which is held or positioned against the body of the wearer during use by a fastening system, such that the article catches the bodily exudates. Typical absorbent articles include a topsheet facing the wearer, which permits fluid exudates to pass through, and a backsheet, which prevents the exudates from escaping from the absorbent article. Although these types of absorbent articles may be highly efficient for the absorption of liquids, it is well recognized that long-term wear of such articles provides a moist, occluded environment and may lead to skin which is compromised in terms of being over hydrated or exposed to skin irritants commonly found in body exudates. It is generally known that skin under absorbent articles is more susceptible to skin disorders, including diaper rash, erythema (i.e., redness), heat rash, abrasion, pressure marks and skin barrier loss.

Recent advances in the manufacture of disposable absorbent articles include the introduction of skin care compositions applied to the skin contacting surfaces of the article. These compositions comprise an emollient to provide a skin benefit, i.e., improve appearance, enhance lubrication, reduce flaking, increase water content, restore suppleness, and retard irritant skin contact, etc., upon transfer from the article to the skin. These compositions typically contain other functional ingredients to enhance the performance of the emollient. Imnmobilizing agents, which function either to increase the viscosity or hardness of the composition, are employed to reduce the flow of the skin care composition into regions of the article which do not contact the skin. For example, immobilizing agents incorporated into the skin care composition to increase its viscosity can retard the composition's flow into the core. For an absorbent article such as diapers, the transfer efficiency of the skin care composition is enhanced for compositions residing directly on the top surface layer of the diaper topsheet, rather than within the diaper core. A skin care composition within the core of the diaper is not readily available for transfer to the baby's skin. In fact, flow of the skin care composition into the core could ultimately lead to severe absorbency problems for articles such as diapers, feminine hygiene products and incontinence pads. This is because many of the ingredients in skin care compositions are hydrophobic in nature and, after coating portions of the core, these ingredients would inhibit absorption of aqueous fluids into the interior of the core. Hardening agents incorporated into skin care compositions can also function to aid in stability and to increase transfer to the skin.

Other ingredients that may be included in the composition include surfactants, emulsifiers, wetting agents, suspending agents, antimicrobial agents, colorants, film formers, and skin active ingredients which may include drugs, vitamins, and botanical extracts.

Although ingredients such as viscosity increasing agents and hardening agents can be incorporated into the skin care composition to increase its transfer to the skin, transfer performance has never reached its full potential, i.e., the skin care composition is never transferred completely to the wearer's skin. Ideally, 100% of the skin care composition from the absorbent article transfers to the skin during use. The transfer of the skin care composition from an absorbent article to the wearer's skin typically occurs via abrasion and frictional forces. In some cases, heat from the skin can also enhance the transfer of the skin care composition via “melting” of beneficial ingredients within the composition. In addition, if a surfactant, wetting agent, or an emulsifier is included in the skin care composition, moisture from the skin, urine or feces could emulsify the skin care composition onto the skin of the wearer. Improved skin transfer of the composition to the wearer's skin could lead to a multitude of benefits: 1) improved skin health, 2) reduced costs, 3) decreased need for a high concentration of expensive skin care ingredients in the composition, and, as has now surprisingly been discovered, 4) improved diaper leakage and fit properties. Thus, it would be desirable to provide skin care compositions having improved transfer properties and adhesion to the skin.

### BACKGROUND ART

The following references relate to skin care compositions: U.S. Pat. No. 6,316,524, issued Nov. 13, 2001, in the names of Corzani et al.; U.S. Pat. No. 6,017,520, issued Jan. 25, 2000, in the names of Synodis et al.; U.S. Pat. No. 5,695,772, issued Dec. 9, 1997, in the name of Kanga et al.; U.S. Pat. No. 5,154,919, issued Oct. 13, 1992, in the name of Desgarets; EPO987008, published Sep. 6, 1999, in the names of Gers-Berlag et al,; JP 02/0721107, published Mar. 13, 1990, assigned to Nippon Oils & Fats Co. Ltd.; JP 57/169414, published Oct. 19, 1982, assigned to Tokyo Eizai Kenkyu,

### SUMMARY OF THE INVENTION

The present invention is directed to a skin care composition which provides enhanced transfer of the composition to the skin and increased adhesion to the skin. The skin care composition comprises:

a) from about 5% to about 95% by weight of an emollient,

b) from about 10% to about 50% by weight of a tackifying agent, and

c) optionally, from about 1% to about 95% by weight of an immobilizing agent,

Skin care compositions according to the present invention have tack values of from about 50 grams to about 350 grams at temperatures of 19 to 23° C. at 8 seconds. Preferred embodiments of the skin care composition comprise 20 to 50% by weight of the tackifying agent.

Due to their increased adhesion, the skin care compositions disclosed herein have improved transfer properties from solid substrates when said compositions are releasably combined with such substrates. They are, therefore, suitable for application to absorbent articles, such as diapers, disposable briefs, disposable swimming pants, adult incontinence garments, feminine hygiene products, and bandages, and to application devices such as cosmetic pads, sponges, patches, and sheet substrates where their increased transfer to the skin and adhesion thereon provide equivalent skin care benefits at lower usage rates than previously available absorbent articles.

Further, the skin care compositions disclosed herein can also be applied to the skin from canisters, stick casings, and aerosols, where their improved adhesion to the skin also results in skin care benefits at lower usage rates than currently marketed products.

### DETAILED DESCRIPTION OF THE INVENTION

All percentages and ratios used herein, unless otherwise indicated, are by weight and all measurements made are at 25° C., unless otherwise designated. The invention hereof can comprise, consist of, or consist essentially of, the essential as well as optional ingredients and components described therein.

Other than in the operating examples, or where otherwise indicated, all numbers expressing quantities of ingredients or reaction conditions used herein are to be understood as modified in all instances by the term “about.”

As used herein, the term “wearable article” refers to articles adapted to be applied or affixed to, or otherwise associated with a portion of a wearer's anatomy for a certain period of time, and often during a wearer's normal activities. Wearable articles may encircle or at least partially enclose a portion of a wearer's body, such as in the case of belts, diapers, training pants, underwear, and the like. Such wearable articles may include elastically extensible and/or fastening components to ensure a proper fit to the wearer and/or fastening components to provide for convenient application and removal of the article from the wearer by a caregiver. Alternatively, in addition to the above-described features, at least a portion of the wearable article may be adhesively affixed to the skin of the wearer. In some embodiments, the wearable article may include a separate element, such as an insert, affixed to or associated with the wearable article. Alternatively, the wearable article may comprise an insert adapted to be attached to or associated with a durable or semi-durable article of clothing, such as underwear or a diaper cover.

As used herein, the term “absorbent article” refers to devices which absorb and contain body exudates and, more specifically, refers to devices that are placed against or in proximity to the body of the wearer to absorb and contain the various exudates discharged from the body.

The term “disposable” is used herein to describe absorbent articles that generally are not intended to be laundered or otherwise restored or reused as absorbent articles, i.e., they are intended to be discarded after a single use and, preferably, to be recycled, composted or otherwise discarded in an environmentally compatible manner.

A “unitary” absorbent article refers to absorbent articles which are formed of separate parts united together to form a coordinated entity so that they do not require separate manipulative parts like a separate holder and/or liner. A preferred absorbent article embodiment of the present invention is a unitary disposable absorbent article, such as a diaper.

As used herein, the term “diaper” refers to an absorbent article generally worn by infants and incontinent persons about the lower torso.

As used herein the term “tack” refers to a property of an adhesive that enables it to form a bond of measurable strength immediately after adhesive and adherent are brought into contact under pressure.

As used herein, the term “tackifier” refers to a compounding material used to enhance the property which causes surfaces to adhere to each other.

As used herein the term “substrate” refers to an underlying support, especially a sheet-like material. Suitable materials for use as substrates in the instant articles are preferably selected from the group consisting of, but not limited to, sheets, typically flexible sheets or batts comprising nonwovens, wovens, sponges, polymeric netted meshes, formed films, battings, and combinations thereof. In preferred embodiments, the substrates may be composite materials such that they each consist of one or more plies, each ply being made from the same or different materials than the other plies.

The present invention is directed to a tacky skin care composition that may, in one embodiment, be used in conjunction with an article, including, but not limited to, disposable absorbent articles. Tackiness of the skin care composition is achieved by incorporating a tackifying agent. The tackifying agent increases the stickiness of the composition and thus promotes increased adhesion to the skin. This increased adhesion leads to increased transfer of the composition to the skin. Many such articles are designed to have bands which encircle the user's waist region (waist bands) or upper thighs (leg bands), generally near the genital regions. By the present invention, it has now been found that, a secondary benefit of this increased tackiness, where the tackiness is sufficient to adhere portions of the absorbent article, itself, to the skin, is to improve containment of body exudates. If these highly tacky compositions are strategically applied within the absorbent article, so as to form a seal around the waist, anal and/or genital regions of a wearer, they can reduce leakage of exudates in those regions where the composition contacts the skin. In addition, these tacky compositions can increase the anchoring of the absorbent article to the skin such that sagging is reduced and an improved fit is achieved. The tacky composition could also be used to increase the skin adhesion of the skin-contacting face of a pocket manufactured within the absorbent article. The pocket is used as holder for auxiliary functional devices such as sensors or secondary absorbent structures. As the opposite face of the pocket is pulled by the caregiver, easier and wider opening of the pocket could be achieved via use of the tacky composition which maintains good adhesion between the article and the skin. This would lead to easier insertion of the auxiliary device within the pocket.

The primary purpose of including skin care compositions in an absorbent article is to improve skin health. Prior articles utilized simple compositions containing only an emollient like petrolatum mixed with a viscosity increasing agent and hardener such as a fatty alcohol like stearyl alcohol and a wax like paraffin or ozokerite. These compositions are applied to the topsheet or other skin-contacting surface of an absorbent article, such as a diaper, and transferred to the skin through abrasion and frictional forces. As noted, heat and moisture can also enhance the transfer of the composition to the skin. Even with low composition transfer amounts from the topsheet to the skin, skin benefits can be realized, especially if the composition contains an additional skin beneficial ingredient such as zinc oxide. Some of these skin benefits are likely due to the occlusive properties of the petrolatum deposited onto the skin. The occlusive layer of petrolatum on the baby's diapered skin both maintains moisture within the skin and prevents irritants from the urine and feces from contacting the skin. In addition, the lubricating nature of the petrolatum reduces irritations that may occur through frictional and abrasive forces present during diaper wear. Transfer of zinc oxide to the skin leads to increased skin protection against diaper rash.

More complex formulations, with additional material handling and skin benefits were developed. For instance, PAMPERS® brand disposable diapers from The Procter and Gamble Company, Cincinnati, Ohio, U.S.A., introduced an ointmented diaper containing a composition which included both the previously noted petrolatum and stearyl alcohol, and zinc oxide. The zinc oxide is commonly used in hand applied diaper creams and salves to prevent and mitigate those symptoms associated with diaper rash. Thus, PAMPERS® brand disposable diapers provided the occlusive barrier protection via the transfer of petrolatum while also soothing diaper rash symptoms via the inclusion of zinc oxide. Due to the high density of the zinc oxide, both suspending agents and wetting agents also had to be incorporated into the composition to maintain ointment homogeneity.

An ongoing challenge of skin care composition containing articles such as diapers is increasing the transfer amounts of the composition from the article to the skin. Transfer can be optimized to a certain extent by optimizing the hardness and viscosity of the formula. For instance, dimethicone fluid is a popular emollient due to several advantageous properties: luxurious feel, efficient spreadability, skin barrier and rash protection, as well as breathability. Unfortunately, many of the most effective dimethicone fluid emollients are liquids at room temperature and would simply diffuse into the diaper core if applied to the topsheet. However, hardening agents can be mixed with the dimethicone fluid emollient to raise both its viscosity and hardness. Many silicone waxes are now available which can be used for this dual purpose of increasing the viscosity and hardness of the composition. It is important to note the hardness and viscosity of the formula must be optimized for both transfer and stability. If the composition is too hard and viscous, transfer to the skin suffers. This can be illustrated by envisioning the difficulty of transferring a hard candle wax to the skin. Conversely, rigidity is among the properties required to maintain stability on a surface of an absorbent article such as a diaper. As an example, the needle penetration hardness (method ASTM D-937) and melting point (method ASTM D-127) of Crompton's W835 microcrystalline wax are 15-20 and 180-190 F., respectively. The fairly low needle penetration range and high melting point range would add in high temperature stability but would hinder transfer to the skin. Conversely, the cone penetration hardness (method ASTM D-937) and melting point (method ASTM D-127) of Crompton's White Protopet 1s are 180-210 and 130-140 F, respectively. The fairly high cone penetration range and low melting point range would aid transfer to the skin but high temperature stability would suffer. Compositions of this invention will have penetration values of between about 1 mm (cone penetration hardness) and about 250 mm (needle penetration hardness) (both determinations are ASTM D-937) and melting points between about 110° F. and about 220° F. In summary, it is important to optimize the hardness and viscosity of the composition to achieve the right balance between the stability of the composition after application to the absorbent article and its ability to transfer to the skin.

The optimization of the properties for both efficient transfer from an article to the skin and stability limits the amount of the composition that can ultimately be transferred to the skin. Soft and low melting compositions lead to excellent skin transfer and poor stability while hard and high melting compositions lead to poor skin transfer and excellent stability. Ideally, 100% of the composition would be transferred to the skin to provide the ultimate in skin health at an economical price. Prior technologies have precluded achieving the goal of improved skin composition transfer efficiencies while maintaining product stability.

Tackifiers

It has now been discovered that a means of maintaining stability while increasing transfer to the skin is to incorporate tackifiers into the skin care composition. The tackifier increases the stickiness of the composition and thus its adhesion to skin. Thus, the probability of transfer of the composition from the surface of an absorbent article, such as a diaper topsheet, to the skin of a wearer is increased. Preferred materials used as tackifiers in the instant skin care compositions are microcrystalline waxes, polybutenes having a kinematic viscosity (ASTM D445) of 100 to 45,000, polyisobutylenes of a Flory Viscosity Molecular Weight of 40,000 to 2,400,000, rosin resins such as those derived from gum rosin, wood rosin, and tall oil rosin, and hydrocarbon resins such as the C-5 aliphatic resins, the C-9 aromatic resins and the dicyclopentadiene cycloaliphatic hydrocarbon resins and mixtures thereof. In addition to the probability of increased lotion transfer to the skin, these tacky compositions can also function to inhibit leakage outside of the absorbent article by creating a hydrophobic seal between the skin and the absorbent article. These compositions are very effective in providing both a skin and leakage benefit if applied to the cuffs of the article. As noted, fit benefits may also be achieved by allowing for anchoring of the absorbent article to the wearer's skin.

It was already noted how optimization of the hardness and viscosity of the composition must occur to balance stability and skin transfer. Similar optimization phenomena also occur with the use of tackifiers where adhesion to the skin can be so high as to cause potential pain during removal of the article. Thus, the tack must be optimized for improved skin transfer efficiencies from the article to the skin while not causing pain upon removal. The tack should not exceed 350 grams as measured under 100 grams of force held for 8 seconds as described in the to be described Texture Analyzer Tack Measurement Method.

The simplest example of a composition which would exhibit increased transfer due to its inherent tackiness is microcrystalline wax. Many microcrystalline waxes are sticky at room temperature and thus would effectively adhere to the skin. With this adhesion to the skin, an effective seal could be formed in regions where the microcrystalline wax contacts the baby's skin. If properly designed, leakage of body exudates from the absorbent article could be reduced and enhanced anchoring might be achieved for improved fit. Although adhesion could be made to the skin via the use of only a microcrystalline wax, actual transfer of the wax to the baby's skin would likely be limited due to the solid nature and high melting point of many of these microcrystalline waxes. Conversely, if liquids could somehow be suspended directly on the surface of the topsheet, they would be the most effective phase for skin transfer. Although gelled liquid mineral oils are available from commercial suppliers, there are still challenges in optimizing transfer and stability properties with their use in absorbent structures. But, tackifiers could be added to these gelled compositions to improve the transfer from the substrate to the skin. Since utilizing current technologies, liquids can't be reasonably suspended on a surface, such as the topsheet of an absorbent article, a compromise must be achieved in balancing the use of liquids and solids in the microcrystalline wax.

As noted, a pure microcrystalline wax would lead to increased adhesion to skin but relatively poor transfer to the skin due to the solid nature of the wax. In the present invention, a composition with the improved balance of both skin transfer and skin adhesion includes a mixture of the microcrystalline wax to function as the tackifier and a liquefiable emollient such as mineral oil or petrolatum to function as the emollient and melt point reducer. The melt point range for these compositions should be in the range of 110-220° F. as measured with ASTM D-127, more preferably in the range of 120-170° F. as measured by ASTM D-127, and most preferably in the range of 130-160° F. as measured by ASTM D-127. Here, the microcrystalline wax tackifier would be employed in the composition to increase the stickiness and thus the adhesion of the composition to the skin. It would also increase the viscosity of the molten composition and the hardness of the formula on the substrate. Once increased adherence to the skin is achieved by the microcrystalline wax addition, the transfer of the mineral oil or petrolatum emollient to the skin becomes more efficient. Without being bound by theory, this increased transfer is suspected to be due to intimacy of contact between the skin care composition and the skin as a consequence of the tackifier inclusion in the formula.

A preferred formula contains petrolatum to function as the skin emollient, and both microcrystalline wax and polyisobutylene as the tackifiers. The microcrystalline wax would function as both a tackifier and hardening agent for the petrolatum. Both the microcrystalline wax and the polyisobutylene materials would also function as viscosity increasing agents in the composition such that at the elevated temperatures where the formula is molten, the increased viscosity inhibits flow of the skin care composition into the core of an absorbent article such as a diaper. This leads to improved compositional stability and more of the composition is available at the top-most surface regions of substrate, such as a diaper topsheet, for most effective transfer to the skin. As noted previously, any skin care composition wasted in the core simply adds to product costs, lowers the transfer efficiency to the skin, and may actually inhibit core absorbency performance.

Emollients

For compositions designed to provide a skin benefit, a required active ingredient is one or more skin protectants or emollients. As used herein, the term “emollient” is a material that protects against wetness or irritation, softens, soothes, supples, coats, lubricates, moisturizes, reduces flaking, protects and/or cleanses the skin. In a preferred embodiment, these emollients will have either a plastic or liquid consistency at ambient temperatures, i.e., 20° C. This particular emollient consistency allows the composition to impart a soft, lubricious, lotion-like feel.

Representative emollients used in the present invention include, but are not limited to, emollients that are petroleum-based such as mineral oil and petrolatum; sucrose ester fatty acids; polyethylene glycol and derivatives thereof; humectants; emollient esters, for example, fatty acid ester types such as myristyl isostearate; alkyl ethoxylate type; fatty acid ester ethoxylates; fatty alcohol type; sterols and sterol esters, polysiloxane type; propylene glycol and derivatives thereof; glycerin and derivatives thereof, including glycerides, acetoglycerides, and ethoxylated glycerides of C₁₂-C₂₈ fatty acids; triethylene glycol and derivatives thereof; spermaceti, ozokerite, or other waxes; fatty acids; fatty alcohol ether ethoxylates, particularly those having from 12 to 50 carbon atoms in their fatty chain, such as stearic acid; propoxylated fatty alcohols; other fatty esters of polyhydroxy alcohols; lanolin and its derivatives; kaolin and its derivatives; any of the monographed skin care agents listed above and including allantoin, aluminum hydroxide, calamine, cocoa butter, shark liver oil, zinc acetate, zinc carbonate and zinc oxide; or mixtures of these ingredients. Suitable petroleum-based emollients include those hydrocarbons, or mixtures of hydrocarbons, having chain lengths of from about 16 to about 32 carbon atoms. Petroleum based hydrocarbons having these chain lengths include mineral oil (also known as “liquid petrolatum”) and petrolatum (also known as “mineral wax,” “petroleum jelly” and “mineral jelly”). Mineral oil usually refers to less viscous mixtures of hydrocarbons having from about 16 to about 20 carbon atoms. Petrolatum usually refers to more viscous mixtures of hydrocarbons having from about 16 to about 32 carbon atoms. Petrolatum and mineral oil are particularly preferred emollients for compositions of the present invention.

Suitable fatty acid ester type emollients include those derived from C₁₂-C₅₀ fatty acids, preferably C₁₆-C₂₂ saturated fatty acids, and short chain (C₁-C₈, preferably C₁-C₃) monohydric alcohols. Representative examples of such esters include methyl palmitate, methyl stearate, isopropyl laurate, isopropyl myristate, isopropyl palmitate, ethylhexyl palmitate and mixtures thereof. Suitable fatty acid ester emollients can also be derived from esters of longer chain fatty alcohols (C₁₂-C₅₀, preferably C₁₂-C₂₂) and shorter chain fatty acids, e.g., lactic acid, such as lauryl lactate and cetyl lactate.

Suitable alkyl ethoxylate type emollients include C₁₂-C₂₂ fatty alcohol ethoxylates having an average degree of ethoxylation of from about 2 to about 50. Preferably, the fatty alcohol ethoxylate emollient is selected from the group consisting of lauryl, cetyl, and stearyl ethoxylates, and mixtures thereof, having an average degree of ethoxylation ranging from about 2 to about 25. Representative examples of such alkyl ethoxylates include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10), C20-C40 Pareth-10 (a long chain primary alcohol ethoxylate having an average degree of ethoxylation of 10), and steareth-10 (a stearyl alcohol ethoxylate having an average degree of ethoxylation of 10). When employed, these alkyl ethoxylate emollients are typically used in combination with the petroleum-based emollients, such as petrolatum, at a weight ratio of alkyl ethoxylate emollient to petroleum-based emollient of from about 1:1 to about 1:5, preferably from about 1:2 to about 1:4.

Suitable fatty alcohol type emollients include C₁₂-C₅₀ fatty alcohols, preferably C₁₆-C₂₂ fatty alcohols. Representative examples include cetyl alcohol, stearyl alcohol, and behenyl alcohol, and mixtures thereof. When employed, these fatty alcohol emollients are typically used in combination with the petroleum-based emollients, such as petrolatum, at a weight ratio of fatty alcohol emollient to petroleum-based emollient of from about 1:1 to about 1:8, preferably from 1:1 to about 1:5, and most preferably from about 1:1 to about 1:2.

Other suitable types of emollients for use herein include polysiloxane compounds. In general, suitable polysiloxane materials for use in the present invention include those having monomeric siloxane units of the following structure:

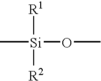

wherein, R¹ and R², for each independent siloxane monomeric unit can each independently be hydrogen or any alkyl, aryl, alkenyl, alkaryl, arakyl, cycloalkyl, halogenated hydrocarbon, or other radical. Any of such radicals can be substituted or unsubstituted. R¹ and R² radicals of any particular monomeric unit may differ from the corresponding functionalities of the next adjoining monomeric unit. Additionally, the polysiloxane can be either a straight chain, a branched chain or have a cyclic structure. The radicals R¹ and R² can additionally independently be other silaceous functionalities such as, but not limited to siloxanes, polysiloxanes, silanes, and polysilanes. The radicals R¹ and R² may contain any of a variety of organic functionalities including, for example, alcohol, carboxylic acid, phenyl, and amine functionalities.

Exemplary alkyl radicals are methyl, ethyl, propyl, butyl, pentyl, hexyl, octyl, decyl, cetyl, octadecyl, and the like. Exemplary alkenyl radicals are vinyl, allyl, and the like. Exemplary aryl radicals are phenyl, diphenyl, naphthyl, and the like. Exemplary alkaryl radicals are toyl, xylyl, ethylphenyl, and the like. Exemplary aralkyl radicals are benzyl, alpha-phenylethyl, beta-phenylethyl, alpha-phenylbutyl, and the like. Exemplary cycloalkyl radicals are cyclobutyl, cyclopentyl, cyclohexyl, and the like. Exemplary halogenated hydrocarbon radicals are chloromethyl, bromoethyl, tetrafluorethyl, fluorethyl, trifluorethyl, trifluorotoyl, hexafluoroxylyl, and the like.

The useful viscosity of the polysiloxanes may vary as widely as the viscosity of polysiloxanes in general vary, so long as the polysiloxane is flowable or can be made to be flowable for application to the article. This includes, but is not limited to, viscosity as low as 5 centistokes (at 37° C. as measured by a glass viscometer) to about 20,000,000 centistokes. Preferably the polysiloxanes have a viscosity at 37° C. ranging from about 5 to about 5,000 centistokes, more preferably from about 5 to about 2,000 centistokes, most preferably from about 100 to about 1000 centistokes. High viscosity polysiloxanes which themselves are resistant to flowing can be effectively deposited upon the article by such methods as, for example, emulsifying the polysiloxane in one or more surfactants or providing the polysiloxane in solution with the aid of a solvent, such as hexane, listed for exemplary purposes only. To enhance the stability of the polysiloxane emollient on the article, silicone waxes may be solubilized into the polysiloxane to raise the melt point and viscosity of the composition.

Preferred polysiloxane compounds for use in the present invention are disclosed in U.S. Pat. No. 5,059,282 (Ampulski et al.), issued Oct. 22, 1991. Particularly preferred polysiloxane compounds for use as emollients in the compositions of the present invention include phenyl-functional polymethylsiloxane compounds (e.g., Dow Corning 556 Cosmetic-Grade Fluid: polyphenylmethylsiloxane) and cetyl or stearyl functionalized dimethicones such as Dow 2502 and Dow 2503 polysiloxanes, respectively. In addition to such substitution with phenyl-functional or alkyl groups, effective substitution may be made with amino, carboxyl, hydroxyl, ether, polyether, aldehyde, ketone, amide, ester, and thiol groups. Of these effective substituent groups, the family of groups comprising phenyl, amino, alkyl, carboxyl, and hydroxyl groups are more preferred than the others; and phenyl-functional groups are most preferred.

When present, the amount of emollient that can be included in the composition will depend on a variety of factors, including the particular emollient involved, the lotion-like benefits desired, costs, and compatibility with the other components in the composition and like factors. The composition will comprise from 5 to about 95%, by total weight, of the emollient. Preferably, the composition will comprise from about 20 to about 80%, more preferably from about 40 to about 75%, by weight, of the emollient.

Immobilizing Agents

Depending on the essential emollient and tackifier chosen, other materials may be included in the composition as immobilizing agents. Accordingly, another component of the therapeutic/skin protective/skin conditioning compositions useful in the methods of the present invention is an optional, additional agent, different from the tackifying agent, which is capable of immobilizing the composition (including the preferred emollient and/or other skin conditioning/therapeutic/protective agents) in the desired location in or on the treated article. Because certain of the preferred emollients in the composition have a plastic or liquid consistency at 20° C., they tend to flow or migrate, even when subjected to modest shear. When applied to a wearer-contacting surface or other location of an absorbent article, especially in a melted or molten state, the emollient will not remain primarily in or on the treated region. Instead, the emollient will tend to migrate and flow to undesired regions of the article.

The immobilizing agent counteracts this tendency of the emollient to migrate or flow by keeping the emollient primarily localized on the surface or in the region of the article to which the composition is applied. This is believed to be due, in part, to the fact that the immobilizing agent raises the melting point and/or viscosity of the composition above that of the emollient. Since the immobilizing agent is preferably miscible with the emollient (or solubilized in the emollient with the aid of an appropriate emulsifier or dispersed therein), it entraps the emollient on the surface of the article's wearer contacting surface or in the region to which it is applied. It should be noted that the category of immobilizing agents would include those classes of ingredients also called viscosity increasing agents, hardening agents and suspending agents since all of these types of ingredients either raise the composition's viscosity or its hardness or both of these properties.

It is also advantageous to “lock” the immobilizing agent on the article's wearer contacting surface or the region of the article to which it is applied. This can be accomplished by using immobilizing agents which quickly set up (i.e., solidify) upon application to the article. Those materials which can act as nucleation sites and set up quickly are typically crystalline and well ordered in structure such as paraffin wax and fatty alcohols. In addition, outside cooling of the treated article via blowers, fans, cold rolls, etc. can speed up crystallization of the immobilizing agent.

In addition to being miscible with (or solubilized in) the emollient, the immobilizing agent will preferably have a melting profile that will provide a composition that is solid or semisolid at ambient temperature. In this regard, preferred immobilizing agents will have a melting point of at least about 35° C. This is so the immobilizing agent itself will not have a tendency to migrate or flow. Preferred immobilizing agents will have melting points of at least about 40° C. Typically, the immobilizing agent will have a melting point in the range of from about 50° to about 150° C.

When utilized, immobilizing agents useful herein can be selected from any of a number of agents, so long as the preferred properties of the skin care composition provide the skin benefits described herein. Preferred immobilizing agents will comprise a member selected from the group consisting of C₁₄-C₂₂ fatty alcohols, C₂₃-C₆₀ alcohols, C₁₄-C₂₂ fatty acids, C₂₃-C₆₀ carboxylic acids, C₁₂-C₂₂ fatty alcohol ethoxylates, and C₂₃-C₆₀ alcohol ethoxylates having an average degree of ethoxylation ranging from 2 to about 40, and mixtures thereof. Preferred immobilizing agents include C₁₆-C₂₂ fatty alcohols, most preferably crystalline high melting materials selected from the group consisting of cetyl alcohol, stearyl alcohol, behenyl alcohol, and mixtures thereof (the linear, crystalline structure of these materials can speed up solidification on the treated absorbent article). Mixtures of cetyl alcohol and stearyl alcohol are particularly preferred. Other preferred immobilizing agents include C₁₆-C₂₂ fatty acids, most preferably selected from the group consisting of palmitic acid, stearic acid, behenic acid and mixtures thereof. Mixtures of palmitic acid and stearic acid are particularly preferred. Still other preferred immobilizing agents include C₁₆-C₂₂ fatty alcohol ethoxylates having an average degree of ethoxylation ranging from about 5 to about 20. Preferably, the alkyl portion of the fatty alcohols, fatty acids and fatty alcohols are linear. Importantly, these preferred immobilizing agents such as the C₁₆-C₂₂ fatty alcohols both increase the rate of crystallization of the composition causing the composition to crystallize rapidly onto the surface of the substrate.

Other types of ingredients that can be used as immobilizing agents, either alone, or in combination with the above-mentioned immobilizing agents, include waxes such as carnauba, ozokerite, beeswax, candelilla, paraffin, microcrystalline, silicone, polyethylene, polyethylene glycol and other polyethylene derivatives, ceresin, esparto, ouricuri, rezowax, isoparaffin, and other known mined, mineral, natural and synthetic waxes. The high melt point of these materials can help immobilize the composition on the desired surface or location on the article. Due to the crystalline, linear structure of paraffin waxes, they can aid in quickly solidifying the composition onto the topsheet after the molten composition is applied. Additionally, microcrystalline waxes can, in addition to acting as tackifiers, be effective immobilizing agents by raising the viscosity and melt point of the composition. Microcrystalline waxes can aid in “locking” up low molecular weight hydrocarbons within the skin care composition. An example of a particularly preferred immobilizing agent, that is also a tackifier is a microcrystalline wax, such as W835 from Crompton Inc, Greenwich, Conn.

Other agents which can function as immobilizing agents include bentonite and hectorite clays and modified derivatives such as those materials from Rheox, silica and modified silicas such as TS-720 from Cabot, Inc, C20 to C60 alcohols such as those marketed by New Phase Inc (Piscataway, N.J.), C24-C45 Alkyl Methicones such as those sold by Dow Corning (Midland, Mich.) and General Electric (Waterford, N.Y.), pentaerythrityl distearate, pentaerythrityl rosinate, polybutenes, polyisobutylenes, and various rosins and rosin esters. Polybutenes, polyisobutylenes, and some rosins can, like the microcrystalline waxes, function as both immobilizing agents and tackifiers.

Suitable polyhydroxy fatty acid esters for use in the present invention will have the formula:

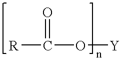

wherein R is a C₅-C₃₁, hydrocarbyl group, preferably straight chain C₇-C₁₉ alkyl or alkenyl, more preferably straight chain C₉-C₁₇ alkyl or alkenyl, most preferably straight chain C₁₁-C¹⁷ alkyl or alkenyl, or mixture thereof; Y is a polyhydroxyhydrocarbyl moiety having a hydrocarbyl chain with at least 2 free hydroxyls directly connected to the chain; and n is at least 1. Suitable Y groups can be derived from polyols such as glycerol, pentaerythritol; sugars such as raffinose, maltodextrose, galactose, sucrose, glucose, xylose, fructose, maltose, lactose, mannose and erythrose; sugar alcohols such as erythritol, xylitol, maltitol, mannitol and sorbitol; and anhydrides of sugar alcohols such as sorbitan.

One class of suitable polyhydroxy fatty acid esters for use in the present invention comprises certain sorbitan esters, preferably the sorbitan esters of C₁₆-C₂₂ saturated fatty acids. Because of the manner in which they are typically manufactured, these sorbitan esters usually comprise mixtures of mono-, di-, tri-, etc. esters. Representative examples of suitable sorbitan esters include sorbitan palmitates (e.g., SPAN 40), sorbitan stearates (e.g., SPAN 60), and sorbitan behenates, that comprise one or more of the mono-, di- and tri-ester versions of these sorbitan esters, e.g., sorbitan mono-, di- and tri-palmitate, sorbitan mono-, di- and tri-stearate, sorbitan mono-, di and tri-behenate, as well as mixed tallow fatty acid sorbitan mono-, di- and tri-esters. Mixtures of different sorbitan esters can also be used, such as sorbitan palmitates with sorbitan stearates. Particularly preferred sorbitan esters are the sorbitan stearates, typically as a mixture of mono-, di- and tri-esters (plus some tetraester) such as SPAN 60, and sorbitan stearates sold under the trade name GLYCOMUL-S by Lonza, Inc. Although these sorbitan esters typically contain mixtures of mono-, di- and tri-esters, plus some tetraester, the mono- and di-esters are usually the predominant species in these mixtures.

Another class of suitable polyhydroxy fatty acid esters for use in the present invention comprises certain glyceryl monoesters, preferably glyceryl monoesters of C₁₆-C₂₂ saturated fatty acids such as glyceryl monostearate, glyceryl monopalmitate, and glyceryl monobehenate. Again, like the sorbitan esters, glyceryl monoester mixtures will typically contain some di- and triester. However, such mixtures should contain predominantly the glyceryl monoester species to be useful in the present invention.

Another class of suitable polyhydroxy fatty acid esters for use in the present invention comprise certain sucrose fatty acid esters, preferably the C₁₂-C₂₂ saturated fatty acid esters of sucrose. Sucrose monoesters and diesters are particularly preferred and include sucrose mono-and di-stearate and sucrose mono- and di- laurate.

Suitable polyhydroxy fatty acid amides for use in the present invention will have the formula:

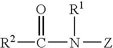

wherein R¹ is H, C₁-C₄ hydrocarbyl, 2-hydroxyethyl, 2-hydroxypropyl, methoxyethyl, methoxypropyl or a mixture thereof, preferably C₁-C₄ alkyl, methoxyethyl or methoxypropyl, more preferably C₁ or C₂ alkyl or methoxypropyl, most preferably C₁ alkyl (i.e., methyl) or methoxypropyl; and R² is a C₅-C₃₁ hydrocarbyl group, preferably straight chain C₇-C₁₉ alkyl or alkenyl, more preferably straight chain C₉-C₁₇ alkyl or alkenyl, most preferably straight chain C₁₁-C₁₇ alkyl or alkenyl, or mixture thereof; and Z is a polyhydroxyhydrocarbyl moiety having a linear hydrocarbyl chain with at least 3 hydroxyls directly connected to the chain. See U.S. Pat. No. 5,174,927 (Honsa), issued Dec. 29, 1992, which discloses these polyhydroxy fatty acid amides, as well as their preparation.

The Z moiety preferably will be derived from a reducing sugar in a reductive amination reaction; most preferably glycityl. Suitable reducing sugars include glucose, fructose, maltose, lactose, galactose, mannose, and xylose. High dextrose corn syrup, high fructose corn syrup, and high maltose corn syrup can be utilized, as well as the individual sugars listed above. These corn syrups can yield mixtures of sugar components for the Z moiety.

The Z moiety preferably will be selected from the group consisting of —CH₂—(CHOH)ₙ—CH₂OH, —CH(CH₂OH)—[(CHOH)ₙ₋₁]—CH₂OH, —CH₂OH—CH₂—(CHOH)₂(CHOR³) (CHOH)—CH₂OH, where n is an integer from 3 to 5, and R³ is H or a cyclic or aliphatic monosaccharide. Most preferred are the glycityls where n is 4, particularly —CH₂—(CHOH)₄—CH₂OH.

In the above formula, R¹ can be, for example, N-methyl, N-ethyl, N-propyl, N-isopropyl, N-butyl, N-2-hydroxyethyl, N-methoxypropyl or N-2-hydroxypropyl. R² can be selected to provide, for example, cocamides, stearamides, oleamides, lauramides, myristamides, capricamides, palmitamides, tallowamides, etc. The Z moiety can be 1-deoxyglucityl, 2-deoxyfructityl, 1-deoxymaltityl, 1-deoxylactityl, 1-deoxygalactityl, 1-deoxymannityl, 1-deoxymaltotriotityl, etc.

As previously noted, some of the immobilizing agents may require an emulsifier for solubilization in the emollient. Suitable emulsifiers will typically include those having HLB values below about 7. In this regard, the sorbitan esters previously described, such as the sorbitan stearates, having HLB values of about 5 or less have been found useful in solubilizing glucamide immobilizing agents in petrolatum. Other suitable emulsifiers include steareth-2 (polyethylene glycol ethers of stearyl alcohol that conform to the formula CH₃(CH₂)₁₇(OCH₂CH₂)ₙOH, where n has an average value of 2), sorbitan tristearate, isosorbide laurate, and glyceryl monostearate. The emulsifier can be included in an amount sufficient to solubilize the immobilizing agent in the emollient such that a substantially homogeneous mixture is obtained. For example, an approximately 1:1 mixture of N-cocoyl-N-methyl glucamide and petrolatum that will normally not melt into a single phase mixture, will melt into a single phase mixture upon the addition of 20% of a 1:1 mixture of Steareth-2 and sorbitan tristearate as the emulsifier. Addition of a tackifier such as a microcrystalline wax or polyisobutylene can be accomplished in this formula to increase the adhesion to the skin and concomitant transfer.

The amount of the optional immobilizing agent that can be included in the composition will depend on a variety of factors, including the actives (e.g., emollients) involved, the particular immobilizing agent involved, the other components in the composition, whether an emulsifier is required to solubilize the immobilizing agent in the other components, and like factors. When present, the composition will typically comprise from about 1 to about 90% of the immobilizing agent. Preferably, the composition will comprise from about 5 to about 50%, most preferably from about 10 to about 40%, of the immobilizing agent.

Additional Optional Components

Additions to the skin care compositions disclosed herein can be selected from among the following: antimicrobial agents, water, surfactants, skin-conditioning agents and skin protectants, other than emollients, humectants, antioxidants, preservatives, chelating agents, viscosity modifiers, suspending agents, pH buffering and adjusting systems, perfumes, soothing agents, pigments, colorants, disinfectants, antibacterial actives, pharmaceutical actives, film formers, deodorants, opacifiers, astringents, and solvents. Usage levels typically range from 0.01% to 25%, preferably 0.1% to 15% and most preferably from 1 to 10%.

Exemplary Embodiments:

Detailed below are seven skin care formulations. Formulas 1 and 2 are typical skin care formulas. Formulas 3 through 7 are representative of the tacky skin care compositions of this disclosure.

TABLE I
EXAMPLE FORMULAS WITH RECIPES

| FORMULA 1 | % | FORMULA 2 | % | FORMULA 3 | % | FORMULA 4 | % |
|---|---|---|---|---|---|---|---|
| Petrolatum¹ | 58.5 | Petrolatum¹ | 75.0 | Petrolatum¹ | 56.2 | Petrolatum¹ | 74.9 |
| Stearyl | 41.5 | Stearyl | 25.0 | Stearyl | 18.7 | Microcrystalline | 25.1 |
| Alcohol² | | Alcohol² | | Alcohol² | | Wax⁴ | |
| | | | | Vistanex³ | 25.1 | | |

| FORMULA 5 | % | FORMULA 6 | % | FORMULA 7 | % |
|---|---|---|---|---|---|
| Petrolatum¹ | 56.2 | Petrolatum¹ | 56.3 | Petrolatum¹ | 65.0 |
| Microcrystalline | 18.8 | Microcrystalline | 18.7 | Microcrystalline | 25.0 |
| Wax⁴ | | Wax⁴ | | Wax⁴ | |
| Vistanex³ | 25.0 | Indopol⁵ | 25.0 | Silica⁶ | 2.0 |
| | | | | Zinc Oxide⁷ | 8.0 |

Sample Preparation
¹Petrolatum, (White Protopet 1s from Crompton, Petrolia, PA)
²Stearyl Alcohol, (CO1897 from The Procter&Gamble Company, Cincinnati, OH)
³Vistanex Polyisobutylene, (LM-MH-LC from Exxon Mobil Corp., Irving, TX)
⁴W835 Microcrystalline Wax (Crompton, Petrolia, PA)
⁵Indopol (Polybutene from BP Amoco Chemicals, Napierville, IL)
⁶Silica (Cab-O-Sil M-5 from Cabot, Inc, Tuscola, IL)
⁷Zinc Oxide (USP grade ZnO powder from JT Baker, Phillipsburg, NJ)

Examples 1 and 2 may be made by mixing the petrolatum and stearyl alcohol and heating the mixture at 90-110° C. for approximately 1 hour or until completely melted.

Example 3 may be made by mixing the petrolatum and stearyl alcohol and heating the mixture at 90-110° C. for approximately 1 hour or until completely melted. Maintain the heat on this petrolatum/stearyl alcohol premix in the range of 110-120° C. and add the Vistanex®. tackifier. Maintain heating in the range of 110-120° C. and stir until the Vistanex®. is completely dissolved in the petrolatumnstearyl alcohol mixture.

Example 4 may be made by mixing the petrolatum and microcrystalline wax and heating the mixture at 90-110° C. for approximately 1 hour or until completely melted.

Example 5 may be made by mixing the petrolatum and microcrystalline wax and heating the mixture at 90-110° C. for approximately 1 hour or until completely melted. Maintain the heat on this petrolatum/microcrystalline wax premix in the range of 110-120° C. and add the Vistanex® tackifier. Maintain heating in the range of 110-120° C. and stir until the Vistanex® is completely dissolved in the petrolatum/microcrystalline wax mixture.

Example 6 may be made by mixing the petrolatum and microcrystalline wax and heating the mixture at 90-110° C. for approximately 1 hour or until completely melted. Maintain the heat on this petrolatum/microcrystalline wax premix in the range of 90-110° C. and add the Indopol® tackifier. Maintain heating in the range of 90-110° C. and stir until the Indopol® is completely dissolved in the petrolatum/microcrystalline wax mixture.

Example 7 may be made by mixing the petrolatum and microcrystalline wax and heating the mixture at 90-110° C. for approximately 1 hour or until completely melted. Maintain the heat on this petrolatum/microcrystalline wax premix in the range of 90-110° C. and slowly add the silica until wetted out by the molten petrolatum/microcrystalline wax. Shear this mixture at 1600 RPM with an IKA T25 homogenizer (IKA Inc) until a homogeneous mixture results while maintaining the temperature in the range of 90-110° C. Stop the homogenizer and add the correct amount of zinc oxide. Shear this final mixture at 1600 RPM with an IKA T25 homogenizer (IKA Inc) until a homogeneous mixture results while maintaining the temperature in the range of 90-110° C.

The tackifier composition may be applied to a substrate via any means of liquid or semi-liquid application as known in the art, including, but not limited to, slot coating, spraying, bead coating, curtain coating, gravure printing, ink jet printing, and digital printing. Alternatively, the tackifier composition may be a solid or semi-solid material affixed to a substrate via adhesive bonding or hydrogen bonding or any bonding mechanisms known in the art (i.e., entanglement, van der Waals forces, electrostatic forces, etc.). The solidification process may be accelerated via the use of convective mass transport, if evaporation of a solvent is required, or convective or conductive heat transfer, e.g., cooling via air or chilled rolls, etc.

The substrate to which the tackifier composition is applied or otherwise affixed may comprise any one, or a combination, of the structural components of the article, including the backsheet, topsheet, fasteners, absorbent material, etc., or may be a separate element added or applied to the product. The substrate may be a film, nonwoven, woven, or foam material and may comprise synthetic and/or natural materials.

Texture Analyzer Tack Measurement Method

The tack of the skin care compositions and raw materials is measured using the Texture Analyzer Tack Measurement Method described here. For this method, each of the formulas is first made and then melted within a temperature range of 80 to 110° C. The raw materials are also heated in separate containers in this temperature range of 80 to 110° C. The texture analyzer samples are prepared by overfilling the molten formulas or raw materials into 45° cone sample cups made from polymethyl methacrylate and having a diameter of 38 mm. The samples are allowed to set up and reach a temperature of 19-23° C. before acquiring the tack data. After cooling to 19-23° C., the surface of each sample is leveled off with a stainless steel spatula blade so the surface is even in height with the outside containment edges of the 45° cone sample cup.

The TA-XT2i Texture Analyzer, manufactured by Stable Micro Systems (Godalming, Surrey, England), is equipped with a 5 kg load cell from Stable Micro Systems and is used to evaluate the adhesive properties of these formulas. The ¼″ diameter stainless steel compression probe (part number TA-8, Texturetechnogies, Scarsdale, N.Y.) attaches to the cross arm of the texture analyzer and measures the tack properties of the various skin care compositions. Before all measurements and at least every four hours, the texture analyzer is calibrated for both force and distance. A 2 kg standard weight calibrates the force of the load cell and a 100 mm calibrated Webber gauge block is used to confirm the accuracy of the texture analyzer's distance setting. After calibration, the 45° sample cone cup containing the leveled skin care composition is firmly secured into the spreadability rig (Texturetechnogies, Scarsdale, N.Y.) with the two attached screws. The spreadability rig mounts to the base by either screwing it into the texture analyzer base or placing a 2 kg weight on the top of each end of the rig.

For this tack measurement method, the probe travels at 4.0 mm per second toward the sample until the sample surface is detected at 5.0 grams of force. At this point, 100.0 grams of force is constantly applied to the sample surface at a speed of 2.0 mm per second for a set time period of either 0.2 or 8.0 seconds. The shorter set time period of 0.2 seconds allows softer samples such as pest trap adhesives to be analyzed. After the set time period, the probe withdraws to a distance of 16.0 to 64.0 mm from the sample's surface at a rate of 4.0 mm per second. Data points are collected at 500 points per second. Each sample is run 5 times by moving a fresh, untested region of the sample under the probe. The stainless steel probe is thoroughly cleaned using a paper wipe (Kimberly-Clark's Kimwipes EX-L, Neenah, Wis.) after each individual test.

The tack values are presented in Table II where 100.0 grams of force was constantly applied to the sample surface at a speed of 2.0 mm per second for a set time period of either 0.2 or 8.0 seconds. The average and standard deviations are calculated for 5 measurements per sample. The samples whose tack values are shown in Table II are Formulas 1-7 detailed above, in Table I, with pure microcrystalline wax, a Victor Pest Trap and pure Vistanex as comparative examples. The Victor Pest Trap is used to illustrate an extremely tacky value.

TABLE II

| | Tack | | Tack | |
|---|---|---|---|---|
| | (grams) | | (grams) | |
| | Hold for | Standard | Hold | Standard |
| Sample Code | 8.0 s | Deviation | for 0.2 s | Deviation |
| Microcrystalline Wax¹ | 175.3 | 8.5 | 72.0 | 4.5 |
| Victor Pest Trap Adhesive² | Too Soft | Too Soft | 181.6 | 10.4 |
| Vistanex | 384.0 | 19.2 | 198.8 | 1.4 |
| (Exxon LM-MH-LC)³ | | | | |
| Formula 1 | 1.2 | 0.8 | 2.7 | 1.5 |
| Formula 2 | 11.3 | 1.5 | 23.9 | 7.6 |
| Formula 3 | 107.9 | 16.6 | 54.6 | 6.4 |
| Formula 4 | 147.6 | 9.9 | 77.8 | 1.4 |
| Formula 5 | 315.3 | 11.5 | 84.0 | 2.2 |
| Formula 6 | 240.9 | 19.4 | 83.6 | 1.7 |
| Formula 7 | 131.8 | 4.3 | 59.1 | 1.1 |

¹W835 Microcrystalline Wax from Crompton, Petrolia, PA
²Victor Pest Trap (Woodfield Inc. PA)
³Vistanex Polyisobutylene, (LM-MH-LC from Exxon Mobil Corp., Irving, TX)

Measurements were run five times for each sample. For each sample, data from the replicates was averaged and the standard deviations calculated.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
a) from about 5% to about 95% by weight of an emollient,
b) from about 10% to about 95% by weight of a tackifying agent, and
c) optionally, from about 1% to about 95% by weight of an immobilizing agent,
wherein the skin care composition has a tack value from about 50 grams to about 350 grams at a temperature of 19 to 23° C. at 8 seconds.

2. The skin care composition of claim 1 comprising 20 to 50% by weight of the tackifying agent.

3. The skin care composition of claim 1 wherein the tackifying agent is selected from the group consisting of microcrystalline waxes, polybutenes, rosin resins, hydrocarbon resins, and mixtures thereof.

4. The skin care composition of claim 3 wherein the tackifying agent is polyisobutylene.

5. The skin care composition of claim 1 wherein the emollient is selected from the group consisting of: petroleum-based emollients; emollient esters; polyol polyesters; fatty alcohol ethers; sterols and sterol esters, and their derivatives; triglycerides; glyceryl esters; ceramides; silicone and silicones derivatives and mixtures thereof.

6. The skin care composition of claim 5 wherein the emollient esters are fatty acid esters.

7. The skin care composition of claim 1 wherein the immobilizing agent is selected from the group consisting of: waxes, polyhydroxy fatty acid esters, polyhydroxy fatty acid amides, C₁₄-C₂₂ fatty alcohols, C₁₂-C₂₂ fatty acids, C₁₂-C₂₂ fatty alcohol ethoxylates having an average degree of ethoxylation of about 2 to about 30, C₂₃-C₆₀ alcohols, C₂₃-C₆₀ carboxylic acids, and C₂₃-C₆₀ alcohol ethoxylates having an average degree of ethoxylation ranging from 2 to about 40, C₈-C₃₀ acid glyceryl esters, solid polyol polyesters, and mixtures thereof.

8. The skin care composition of claim 1 additionally comprising one or more of the following: water, surfactants, skin care agents, humectants, antioxidants, viscosity modifiers, suspending agents, pH buffering systems, perfumes, soothing agents, pigments, disinfectants, antibacterial actives, pharmaceutical actives, film formers, deodorants, opacifiers, astringents, and solvents.

9. The skin care composition of claim 1 having a penetration value of between about 1 mm (cone penetration hardness) and about 250 mm (needle penetration hardness).

10. The skin care composition of claim 1 having a melting point from about 110° F. to about 220° F.

11. The skin care composition of claim 1 wherein the ratio of emollient to tackifier is from 1:1 to 9:1.

12. The skin care composition of claim 1 comprising a 1:1 to a 1:5 mixture of a tackifier and an emollient to which is added a 1:1 to a 1:5 mixture of an immobilizing agent and an emulsifier.

13. The skin care composition of claim 9 comprising a 1:1 mixture of N-cocoyl-N-methyl glucamide and petrolatum to which is added a 1:1 mixture of Steareth-2 and sorbitan tristearate.

14. An absorbent article selected from the group consisting of diapers, disposable briefs, disposable swimming pants, adult incontinence garments, feminine hygiene products, and bandages comprising the skin care composition of claim 1.

15. An absorbent article according to claim 14 wherein the skin care composition creates a hydrophobic seal between a wearer's skin and the absorbent article.

16. An absorbent article according to claim 14 further comprising cuffs and wherein the skin care composition creates a hydrophobic seal between a wearer's skin and one or more of said cuffs.

17. An absorbent article according to claim 14 comprising a skin care composition comprising:
a) from about 5% to about 95% by weight of an emollient,
b) from about 20% to about 95% by weight of a tackifying agent, and
c) optionally, from about 1% to about 95% by weight of an immobilizing agent,
wherein the skin care composition has a tack from about 50 grams to about 350 grams at a temperature of about 19 to about 23° C. at 8 seconds.

18. An absorbent article according to claim 14 wherein the tackifying agent is selected from the group consisting of microcrystalline waxes, polybutenes, rosin resins, hydrocarbon resins and mixtures thereof.

19. An article to be placed in contact with the skin for applying a skin care composition to the contacted skin, said article comprising a delivery vehicle and a skin care composition releasably disposed on at least a portion of said delivery vehicle, said skin care composition comprising:
a. from about 5% to about 95% by weight of an emollient, selected from the group consisting of: petroleum-based emollients; emollient esters; polyol polyesters; fatty alcohol ethers; sterols and sterol esters, and their derivatives; triglycerides; glyceryl esters; ceramides; silicones and silicone derivatives and mixtures thereof;
b. from about 20% to about 50% by weight of a tackifying agent, selected from the group consisting of: microcrystalline waxes, polybutenes, rosin resins, and hydrocarbon resins, and
c. optionally, from about 5% to about 95% by weight of an immobilizing agent selected from the group consisting of: waxes, polyhydroxy fatty acid esters, polyhydroxy fatty acid amides, C₁₄-C₂₂ fatty alcohols, C₁₂-C₂₂ fatty acids, C₁₂-C₂₂ fatty alcohol ethyoxylates having an average degree of ethoxylation of about 2 to about 30, C₂₃-C₆₀ alcohols, C₂₃-C₆₀ carboxylic acids, and C₂₃-C₆₀ alcohol ethoxylates having an average degree of ethoxylation ranging from 2 to about 40, C₈-C₃₀ acid glyceryl esters, solid polyol polyesters, and mixtures thereof,
and wherein the skin care composition has a tack value of at least about 50 grams at a temperature of about 19 to about 23° C. at 8 seconds.

20. An application device to which the skin care composition of claim 1 is releasably affixed, said device being selected from the group consisting of cosmetic pads, sponges, patches, and sheet substrates, canisters, stick casings, and aerosols.
